(19) United States
(12) Patent Application Publication
(10) Pub. No.: US 2023/0266304 A1
(43) Pub. Date: Aug. 24, 2023

(54) OBESE FERRET MODEL AND METHODS OF ESTABLISHING AND USING THE SAME

(71) Applicant: ST. JUDE CHILDREN'S RESEARCH HOSPITAL, INC., Memphis, TN (US)

(72) Inventors: Stacey SCHULTZ-CHERRY, Memphis, TN (US); Victoria MELIOPOULOS, Memphis, TN (US); Rebekah HONCE, Memphis, TN (US)

(21) Appl. No.: 18/005,105

(22) PCT Filed: Jul. 12, 2021
(86) PCT No.: PCT/US2021/041230

§ 371 Date: Jan. 11, 2023

Related U.S. Application Data
(60) Provisional application No. 63/051,984, filed on Jul. 15, 2020.

(51) Int. Cl. G01N 33/50

(52) U.S. Cl. CPC G01N 33/5088; G01N 2333/11; G01N 2333/165; G01N 2800/52; G01N 2333/46

(57) ABSTRACT

A method for establishing an obese ferret model by feeding a ferret with a diet having at least 25% of carbohydrate content for a period of time to provide the obese ferret model is disclosed as is a method of using said model to screen a substance for treating a respiratory infection.

### INTRODUCTION

This patent application claims the benefit of priority from U.S. Provisional Ser. No. 63/051,984 filed Jul. 15, 2020, the contents of which is incorporated herein by reference in its entirety.

This invention was made with government support under grant nos. AI140766 and HHSN272201400006C awarded by the National Institutes of Health. The government has certain rights in this invention.

### BACKGROUND

Obesity rates have nearly tripled worldwide since 1975. Approximately 1.9 billion people are overweight and over 650 million are obese, defined as having a body mass index (BMI) of 25 to 30 and >30, respectively, which translates to nearly 45% of adults worldwide. The obesity-induced inflammatory state has systemic implications for individual and global public health. It is a well-identified risk factor for increased mortality due to heightened rates of heart disease, certain cancers, and musculoskeletal disorders. Over nutrition, as well as undernutrition, has been cited as an important factor in the body's response to infection for centuries. More recently, the impact of obesity on communicable diseases has been appreciated. During the 2009 influenza A virus (IAV) H1N1 pandemic, a plethora of epidemiologic studies revealed obesity to be an independent risk factor for severe disease. In initial retrospective studies of laboratory-confirmed H1N1 cases after the 2009 pandemic, obesity was identified as a risk factor for hospitalization, the need for mechanical ventilation, and mortality upon infection.

Influenza is a potentially severe respiratory infection caused by the influenza virus. Most human cases are caused by H1N1 and H3N2 IAV strains. Several case studies of severe and fatal IAV infections have identified possible effects of obesity on disease progression; these effects include extensive viral replication in the deep lung, progression to viral pneumonia, and prolonged and increased viral shedding. However, these studies neglected to determine the causality between obesity and severe IAV pathogenesis. Studies in mouse models of obesity, including leptin-deficient (OB) and leptin-receptor-deficient (DB) genetically obese models as well as the high-fat diet-induced-obese (DIO) model have identified several immunological mechanisms for the increased pathogenesis and mortality that mirrors what has been seen in humans. However, the mouse respiratory tract is not equivalent to the human respiratory tract. By comparison, ferrets have a similar influenza receptor distribution as that observed in humans (de Graaf & Fouchier (2014) EMBO J. 33(8):823-841) and closely mimic human influenza, with regards to both the sensitivity to infection and the clinical response.

### SUMMARY OF THE INVENTION

This invention provides a method for establishing an obese ferret model by feeding a ferret with a diet having at least 25% of carbohydrate content for a period of time to provide the obese ferret model. In one embodiment, the obese ferret model exhibits at least one of visceral adiposity, hyperglycemia, or reduced high-density lipoprotein cholesterol compared to a control ferret. In another embodiment, the obese ferret model does not develop insulinoma. An obese ferret model and a method for using the same to screen a substance for treating a respiratory infection, e.g., a respiratory bacterial, fungal or viral (e.g., influenza virus or coronavirus) infection are also provided.

### BRIEF DESCRIPTION OF THE DRAWINGS

FIG. 1A-1F demonstrate differences in weight (FIG. 1A), weight gained (FIG. 1B), skinfold fat (FIG. 1C), circumference (FIG. 1D), length (FIG. 1E) and BMI (FIG. 1F) of ferrets fed a lean high-density ferret diet (Lean) and ferrets fed a high-carbohydrate diet (Obese).

FIG. 2 shows the clinical scores of obese and lean ferrets infected with different strains of influenza (i.e., A/California/04/2009 (pH1N1) and A/Memphis/257/2019 (H3N2)).

FIG. 3 shows increased viral titers in the obese ferret model early in infection (day 2) as compared to lean ferrets. TCID₅₀, 50% Tissue Culture Infective Dose.

FIG. 4 shows an increase in viral spread to deep lung tissue in the obese ferret model compared to those on the lean diet.

### DETAILED DESCRIPTION OF THE INVENTION

Ferrets are lean, muscular obligate carnivores that require a high-fat, protein-rich diet. Given that high-carbohydrate diets induce insulinoma, obesity in ferrets needed to be induced in a controlled manner. Accordingly, a method for establishing an obese ferret model has now been developed. Compared to a lean high-density ferret diet composed of a restricted volume of food including 17% carbohydrates, 36% protein, and 47% fat, the obese diet included a 1:1 mix of the high-density ferret diet plus a feline diet (40% carbohydrates, 32% protein, 27% fat) ad libitum, which was supplemented with wet kitten food once per day. After six to twelve weeks on the obese diet, ferrets exhibited an elevated waist circumference, visceral adiposity, fasting glucose, total cholesterol, as well as elevated ALT, globulin, phosphorus, leptin and total protein levels and reduced high-density lipoprotein cholesterol (HDL-C) compared to ferrets fed a lean diet. Collectively, the co-occurrence of visceral adiposity, hyperglycemia, and reduced HDL-C compared to lean control ferrets indicated the development of MetS in the DIO ferret model. Notably, independent of viral strain, the obese ferrets of this invention suffered increased risk of being symptomatic upon infection. Accordingly, this invention provides an obese ferret model and methods of establishing and using the same to study respiratory infections and treatment of the same.

The obese ferret model of this invention is established by feeding a ferret with a diet having at least 25% of carbohydrate content for a period of time to provide the obese ferret model without inducing insulinoma. The ferrets of this invention may be initially obtained from breeding farms and are preferably male ferrets. In some embodiments, the ferrets have a body weight in the range from 300 to 3000 gram, more preferably in the range from 400 to 2600 gram.

To establish the obese ferret model, ferrets are fed a high-carbohydrate diet. In particular, the ferrets are fed a diet having a carbohydrate content of at least 25%, at least 26%, at least 27%, at least 28% or at least 29% based upon the total dry weight of the animal feed. In certain embodiments, the ferrets are fed a diet having a carbohydrate content of about 28.5% based upon the total dry weight of the animal feed. In some embodiments, the animal feed has a total protein content in the range of 30% to 38%, or more preferably, 32% to 36% or most preferably 34%. In other embodiments, the animal feed has a total fat content in the range of 15% to 50%, or more preferably, 27% to 47% or most preferably 37%. The animal feed may be composed of a single animal feed or a combination of animal feeds. By way of illustration, the Examples herein describe the use of a combination of a high-density ferret diet (17% carbohydrates, 36% protein, 47% fat) and a feline diet (40% carbohydrates, 32% protein, 27% fat) in a 1:1 ratio. Preferably, the animal is initially restricted to a particular volume of food and subsequently feed ad libitum. Moreover, the animal may be provided additional supplemental food, e.g., additional protein and/or fat.

In accordance with the method of this invention, the ferrets are fed a high-carbohydrate diet for a period of time to provide the obese ferret model. Ideally, the high-carbohydrate diet begins one to two weeks prior to weaning and can include supplementation with a conventional milk replacer. Upon weaning, e.g., at 7 weeks of age, the ferrets are preferably fed a restricted volume (e.g., 90 to 135 g) of the high-carbohydrate diet for at least one to two weeks. Subsequently, e.g., at 9 weeks of age, the ferrets are allowed to consume the high-carbohydrate diet ad libitum for at least two weeks and may optionally consume an additional supplemental food, e.g., a restricted volume of wet kitten food (9% protein, 5% fat, 1% fiber). Ideally, the feeding protocol is carried out for at least a total of 4 to 5 weeks to provide the obese ferret model.

The obese ferret model is established once the ferrets exhibit at least one of visceral adiposity, hyperglycemia, or reduced high-density lipoprotein cholesterol compared to a control ferret, i.e., a ferret fed a conventional high-density ferret diet (17% carbohydrate, 32% protein, 47% fat) over the same time period without inducing insulinoma. Alternatively, or in addition to, a ferret is deemed to be obese when the ferret exhibits a significant increase in weight, skinfold fat, circumference and/or BMI compared to a control ferret.

The term “visceral adiposity” refers to a condition with increased visceral fat tissue. Visceral adiposity is typically caused by (accumulation of) excessive visceral fat tissue. Visceral fat, also known as organ fat, intra-abdominal fat, peritoneal fat or central fat, is normally located inside the peritoneal cavity as opposed to subcutaneous fat which is found underneath the skin and intramuscular fat which is found interspersed in skeletal muscles. Visceral fat includes the abdominal fat surrounding the vital organs and includes mesenteric fat, perirenal fat, retroperitoneal fat and preperitoneal fat (fat surrounding the liver). Visceral adiposity may be assessed imaging techniques including computed tomography (CT), magnetic resonance imaging (MRI), ultrasonography (US) or by massing total adipose tissue in the abdominal compartment.

Hyperglycemia refers to a higher than normal fasting blood concentration of glucose. In ferrets, the normal fasted blood glucose level is between 65 mg/dL to 112 mg/dL. Accordingly, a fasted blood glucose level exceeding 112 mg/dL, or more preferably 120 mg/dL, or most preferably 130 mg/dL is considered hyperglycemic.

In some embodiments, the HDL-C levels are reduced in the obese ferret model by at least 3%, 5%, 10%, 15%, 20%, 25%, 30%, 35%, 40%, 45%, or 50% compared to a control ferret.

Once established, the obese ferret model of this invention finds use as a model system for obesity and comorbidities of the same. Such comorbidities include, but are not limited to, insulin resistance and type 2 diabetes mellitus, hypertension, dyslipidemia, cardiovascular disease, stroke, sleep apnea, gallbladder disease, hyperuricemia and gout, osteoarthritis and respiratory diseases such as obstructive sleep apn/ea (OSA), obesity hypoventilation syndrome (OHS) and respiratory infections. In some aspects, the obese ferret model can be used to screen for substances or compounds that prevent or treat obesity or a comorbidity of the same. In accordance with this aspect, the obese ferret model is provided a test substance and the effect of the test substance on the prevention or treatment of obesity or comorbidity is assessed.

In certain aspects, the obese ferret model is used in a method for screening a substance for the prevention or treatment of a respiratory disease, in particular a respiratory infection. This aspect of the invention includes the steps of providing a test substance to an obese ferret model and a control ferret either before or after infecting the obese ferret model and a control ferret with an infectious agent that causes a respiratory infection; and determining whether the test substance prevents or treats the respiratory infection.

As used herein, the phrase “respiratory” encompasses all organs and tissues that are involved in the process of respiration in a human subject or other mammal subject, including cavities connected to the respiratory tract such as ears and eyes. In certain embodiments, the respiratory tract, as used herein, encompasses the upper respiratory tract, including the nose and nasal passages, prenasal sinuses, pharynx, larynx, trachea, bronchi, and nonalveolar bronchioles; and the lower respiratory tract, including the lungs and the respiratory bronchioles, alveolar ducts, alveolar sacs, and alveoli therein.

Respiratory infections that can be assessed in the method of the invention include viral respiratory infections, bacterial respiratory infection and fungal respiratory infections. In this respect, examples of infectious agents are viruses, bacteria and fungi. Viruses that cause respiratory tract infections include rhinoviruses, parainfluenza virus infections (PIV, e.g., PIV-1, PIV-2 or PIV-3), bocaviruses, human metapneumoviruses, respiratory syncytial virus (RSV), influenza viruses A, B or C, coronavirus (e.g., MERS or SARS such as SARS-CoV-2), adenoviruses, reovirus (‘respiratory enteritic orphan virus’), etc. Common respiratory bacterial infections may be caused by bacteria such as Streptococcus pneumonia, Staphylococcus aureus, Haemophilus influenzae, Chlmayda pneumoniae, C. psittaci, C. trachomatis, Moraxella (Branhamella) catarrhalis, Legionella pneumophila, Klebsiella penumoniae, and Mycobacterium tuberculosis. Examples of respiratory infections caused by fungi include systemic candidiasis (e.g., Candida albicans, C. troicalis, or C. glabrata), blastomycosis cryptococcosis (e.g., Cryptococcus neoformans or C. gattii), coccidioidomycosis (e.g., Coccidioides immitis), and aspergillosis (e.g., Aspergillus fumigatus). Respiratory infections may be primary or secondary infections.

The infectious agent may cause a bacterial-, viral- and/or fungal bronchiolitis, a bacterial-, viral- and/or fungal pharyngitis and/or laryngotracheitis, a bacterial-, viral- and/or fungal pneumonia, a bacterial-, viral- and/or fungal pulmonary infection, a bacterial-, viral- and/or fungal sinusitis, a bacterial-, viral- and/or fungal upper and/or lower respiratory tract infection, a bacterial-, viral- and/or fungal-exacerbated asthma, a respiratory syncytial viral infection, or Aspergillosis, aspergilloma, Cryptococcosis, emphysema, otitis, a bacterial-, viral- and/or fungal otitis externa, otitis media, conjunctivitis, uveitis primary ciliary dyskinesia (PCD) and pulmonary aspergillosis.

Substances that can be screened in accordance with this method of the invention include, but are not limited to, antibacterial compounds, antifungal compounds, antiviral compounds, vaccines, small organic compounds, antibodies, inhibitory RNA molecules, and the like. Substances may be screened for efficacy in treating a bacterial, viral or fungal infection or evaluating resistance development against antiviral, antibacterial or antifungal agents. Advantageously, the present obese ferret model provides a prolonged virus shedding if infected with a virus, enabling the possibility to evaluate the efficacy of an antiviral agent as well as the emergence of mutant resistant viruses. Indeed, the instant obese ferret model advantageously enables study of virus shedding, pathogen load, virus pathogenicity, duration of virus shedding and emergence of antiviral resistance.

As used herein, the term “treating” includes abrogating, substantially inhibiting, slowing or reversing the progression of a condition, substantially ameliorating clinical or aesthetical symptoms of a condition, and substantially preventing the appearance of clinical or aesthetical symptoms of a condition, namely preemptive, preventative and prophylactic treatment. In this respect, a substance is deemed to have a therapeutic/prophylactic effect if the animal exhibits a reduction, diminishment or an improvement in at least one symptom of a respiratory infection after being administered said substance. In one example, the improvement in at least one symptom of an infection may include amelioration of coughing, sneezing, etc. For example, after administering a test substance or a combination of test substances, if the infected animal exhibits one or more of decreased mucus production, coughing, bronchoconstriction (i.e., wheezing), fever, sinus pain, lesions in the lung, inflammation of bronchial tubes, and sore throat, it is indicative that the test substance or the combination test substances may have a preventive or therapeutic effect on the symptom of respiratory infection.

The following non-limiting examples are provided to further illustrate the present invention.

### Example 1: Materials and Methods

Ferret Diets and Measurements. Neutered male, 6-week-old ferrets (Mustela putorius furo) were obtained from Triple F Farms (Gillett, Pa.). Immediately upon arrival, ferrets were randomly assigned to the diet-induced obesity (DIO) group or to the lean diet group. Diets were allocated daily per cage of three ferrets according to age and diet group for 12 weeks (Table 1).

TABLE 1

| Weeks of | Weeks | | |
|---|---|---|---|
| Age | on Diet | Lean | Obese |
| 6 | 0 | 135 g ground | 135 g ground |
| | | ferret diet | ferret + |
| | | 15 g milk replacer | feline diet mix |
| | | 150 ml distilled | 15 g milk replacer |
| | | water | 150 ml distilled |
| | | | water |
| 7 | 1 | 135 g ground | 135 g ground |
| | | ferret diet | ferret + |
| | | 150 ml distilled | feline diet mix |
| | | water | 150 ml distilled |
| | | | water |
| 8 | 2 | 90 g ground ferret | 90 g ground ferret + |
| | | diet | feline diet mix |
| | | 90 ml distilled | 90 ml distilled |
| | | water plus | water plus |
| | | 90 g solid ferret | 135 g solid ferret + |
| | | diet | feline mix |
| 9 | 3 | 135 g solid ferret | Ad libitum solid |
| | | diet | ferret + feline mix |
| | | | 37.5 g wet kitten |
| | | | food |
| 10+ | 4+ | 135 g solid ferret | Ad libitum ferret + |
| | | diet | feline mix |
| | | | 75 g wet kitten |
| | | | food |

All ferrets were provided ad libitum access to water. For lean ferrets, a restricted volume of high-density ferret diet (Lab Supply; 17% carbohydrates, 36% protein, 47% fat) was fed once daily in the morning. Obese ferrets received a 1:1 mix of high-density ferret diet plus feline diet (Lab Supply; 40% carbohydrates, 32% protein, 27% fat) ad libitum and supplemented with wet kitten food (Iams) once per day. Diets continued for 12 weeks. Throughout the diet period, ferret measurements were recorded biweekly under sedation with 4% isoflurane. Weight and temperature were recorded weekly without sedation. Ferret body length was measured from nose to base of tail and weight circumference just above the iliac crest. Measures of skinfold fat were determined using a digital fat caliper on right side of the abdomen, again above the iliac crest. Ferret body mass index (BMI) was calculated by standardizing the product of weight in kilograms and circumference in centimeters by the ferret length in centimeters squared.

$$ferret BMI = \frac{weight ( kg ) \times circumference ( cm )}{length ( cm ) \times length ( cm )}$$

Histology. The left lateral liver lobe and right kidney were excised and immediately fixed in 10% buffered formalin. Tissues were dehydrated through a series of increasing ethanol concentrations (70-100% in ddH₂O), then placed in xylene and embedded in paraffin. Sections (8 μm) from each block were stained with hematoxylin and eosin. All slides were microscopically analyzed and images captured. Liver fat accumulation was scored by three trained individuals blinded to diet treatments where grade 0 is no evidence of fat vacuoles, grade 1 is evidence of fat vacuoles in <33% of hepatocytes, grade 2 is evidence of fat vacuoles in 33-66% of hepatocytes, and grade 3 is evidence of fat vacuoles in >66% of hepatocytes (Brunt, et al. (1999) Am. J. Gastroenterol. 94(9):2467-74). Histological evaluation of the right kidney included gross morphological assessment which included the following: glomerular hypercellularity and matrix deposition, interstitial hypercellularity, tubulointerstitial calcification, inflammation, and fibrosis.

Lipid Extraction and Quantification. To determine total lipid content of the liver, lipid extraction was performed according to known methods (Bligh & Dyer (1959) Can. J. Biochem. Physiol. 37(8):911-7). Briefly, one gram of flash frozen liver tissue excised from the right medial liver lobe was homogenized in Tris/EDTA buffer. A chloroform, methanol, acetic acid (2:1:0.15, v/v/v) solution was added to liver samples and centrifuged at 900×g for 10 minutes at 10° C. The bottom chloroform layer was collected and mixed with chloroform:methanol (4:1, v/v), then centrifuged at 900×g at 10° C. for 10 minutes. The chloroform layer was then collected and filtered. Extracted lipids were dried under nitrogen gas and total lipid content in the liver was gravimetrically determined.

Triglyceride content of the liver was determined using a triglyceride colorimetric assay kit according to manufacturer's directions (Cayman Chemical). Briefly, 500 mg of flash frozen liver tissue was excised from the left medial liver lobe and homogenized in NP40 assay reagent with EDTA (1 mM). The homogenate was cleared via centrifugation at 10,000×g for 10 minutes at 4° C., and supernatant diluted 1:10 for analysis. Duplicate wells of standards and samples were added to the plate, activated, and then incubated for 15 minutes at room temperature. Absorbance was measured at 550 nm using plate reader and triglyceride content in livers calculated compared to the standard curve.

Infection Symptom Score. To determine infection severity, ferret weight, temperature, and behavior were recorded daily. A temperature above 40° C. indicates fever (0=temperature less than 39.9° C., 1=temperature greater than or equal to 40.0° C.). Animal behavior was recorded continuously for 60 minutes daily to determine the presence and intensity of the following: sneezing (0=none, 1=mild, 2=severe), lethargy (0=active and playful, 1=active when stimulated, 2-not active when stimulated, 3=lethargic), and coughing (0=absent, 1=present). The following signs of illness were also recorded: nasal discharge (0=absent, 1=present), conjunctivitis (0=absent, 1=present), and nasal wash color and consistency (0=clear, 1=cloudy, 2=discolored, 3=discolored and thick). Nasal wash color was recorded every other day. Total symptom score is the sum of each category.

### Example 2: Biomarkers of Obesity and Metabolic Syndrome and in DIO Ferrets

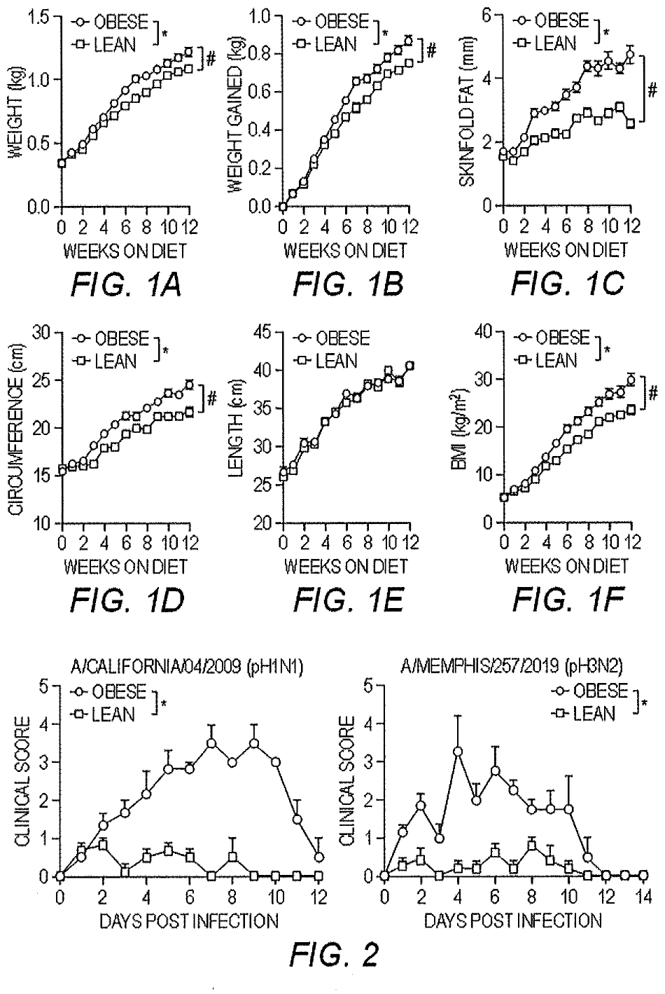

Immune dysfunction due to obesity may stem from obesity's core involvement in triggering metabolic syndrome (MetS) (Andersen, et al. (2016) Adv. Nutr. 7:66-75). The chronic low-grade inflammation implicated as the etiology of poor antiviral responses upon infection with influenza A virus may result from perturbations to cellular metabolism due to MetS (Andersen, et al. (2016) Adv. Nutr. 7:66-75; Easterbrook, et al. (2011) Influenza Other Respir. Viruses 5(6): 418-25; Honce & Schultz-Cherry (2019) Front. Immunol. 10:1071; Karlsson, et al. (2016) MBio. 7(4):e01144-16). The National Institutes of Health (NIH) and National Cholesterol Education Program (NCEP) Adult Treatment Panel III define MetS in adults as the constellation of three of the following five signs: elevated waist circumference, hypertension, fasting hypertriglyceridemia, fasting hyperglycemia, and low high-density lipoprotein cholesterol (HDL-C) levels (Huang (2009) Dis. Models Mech. 2(5-6):231-7). To ascertain the extent of MetS in the obese ferret model (Table 2) as compared to lean ferrets (Table 3), a diagnostic panel of blood chemistries determined plasma levels of albumin, alkaline phosphatase (ALKP), alanine transaminase (ALT), amylase, blood urea nitrogen (BUN), calcium, creatinine, globulin, glucose, phosphorus, potassium, sodium, total bilirubin, total protein, and cholesterol. In addition, weight, weight gained, skinfold fat, circumference, length and BMI were determined (FIG. 1A-1F).

TABLE 2

| | Obese | | |
|---|---|---|---|
| Analyte | Baseline | Midpoint | Final |
| Albumin | 2.63 ± 0.15 | 2.66 ± 0.59 | 3.55 ± 0.35 |
| (g/dL) | | | |
| ALKP (U/L) | 203.25 ± 15.71 | 299.29 ± 111.22 | 127.75 ± 23.57 |
| ALT (U/L) | 123.5 ± 29.46 | 307.38 ± 108.72 | 400.75 ± 184.01* |
| Amylase | 38.25 ± 8.5 | 61.12 ± 31.83 | 33.75 ± 6.45 |
| (U/L) | | | |
| BUN | 30.2 ± 2.8 | 44.62 ± 16.65 | 26.15 ± 8.22 |
| (mg/dL) | | | |
| BUN: | 126.5 ± 45.11 | 115.73 ± 50.4 | 110.85 ± 28.95 |
| Creatinine | | | |
| Calcium | 10.83 ± 0.74 | 7.92 ± 2.84 | 10.95 ± 0.91 |
| (mg/dL) | | | |
| Cholesterol | 172.25 ± 15.09 | 539.17 ± 332.95* | 184.75 ± 19.87 |
| (mg/dL) | | | |
| Creatinine | 0.27 ± 0.11 | 0.41 ± 0.1 | 0.24 ± 0.07 |
| (mg/dL) | | | |
| Globulin | 2.45 ± 0.24 | 4.49 ± 1.93* | 2.6 ± 0.24 |
| (g/dL) | | | |
| Glucose | 112.25 ± 12.84 | 125.14 ± 37.86* | 133.5 ± 24.34*^{‡} |
| (mg/dL) | | | |
| HDL-C | 149.25 ± 13.6 | 140.02 ± 101.55 | 184.25 ± 16.68 |
| (mg/dL) | | | |
| Potassium | 4.95 ± 0.22 | 5.46 ± 0.88^{†} | 5.22 ± 0.12 |
| (mmol/L) | | | |
| LDL-C | 5.26 ± 0.26 | 33.81 ± 40.83 | 144.75 ± 4.27^{‡} |
| (mg/dL) | | | |
| Sodium | 146.0 ± 0.82 | 157.46 ± 23.06^{†} | 149.25 ± 1.26 |
| (mmol/L) | | | |
| NA:K | 29.5 ± 1.29 | 28.90 ± 1.05^{†} | 28.63 ± 0.76* |
| Phosphorus | 9.83 ± 0.82 | 10.65 ± 2.99* | 8.08 ± 0.26 |
| (mg/dL) | | | |
| Total | 0.96 ± 0.17 | 0.64 ± 0.67 | 0.26 ± 0.07 |
| Bilirubin | | | |
| (mg/dL) | | | |
| Protein | 5.08 ± 0.33 | 7.15 ± 2.27* | 6.18 ± 0.25 |
| (g/dL) | | | |
| TAG | 152 ± 54.95 | 118.58 ± 68.35 | 50.75 ± 17.11 |
| (mg/dL) | | | |

All comparisons made between week 0 (baseline), 6 (midpoint), and 12 (final) blood chemistries unless otherwise noted.
*Significant increase in measure from other diet group.
^{†}Comparisons made between week 4 blood chemistries.
^{‡}Comparisons made between week 10 blood chemistries. Analyzed via mixed-effects analysis with Sidak's multiple comparisons test between lean and DIO groups each week.
All data represented as means ± standard deviation and significance set at α = 0.05.

TABLE 3

| | Lean | | |
|---|---|---|---|
| Analyte | Baseline | Midpoint | Final |
| Albumin (g/dL) | 2.83 ± 0.15 | 2.4 ± 0.48 | 3.20 ± 0.18 |
| ALKP (U/L) | 271.0 ± 97.31 | 234.68 ± 57.67 | 114.0 ± 21.95 |
| ALT (U/L) | 165.25 ± 58.91 | 256.61 ± 103.3 | 234.25 ± 73.89 |
| Amylase (U/L) | 42.75 ± 7.89 | 47.62 ± 18.15 | 25.5 ± 4.8 |
| BUN (mg/dL) | 27.45 ± 2.3 | 29.14 ± 13.04 | 25.98 ± 3.94 |
| BUN: | 98.25 ± 28.65 | 77.61 ± 51.54 | 146.83 ± 86.35 |
| Creatinine | | | |
| Calcium | 11.7 ± 1.37 | 6.79 ± 1.71 | 10.08 ± 0.72 |
| (mg/dL) | | | |
| Cholesterol | 155.5 ± 11.21 | 381.31 ± 139.83 | 177.25 ± 8.54 |
| (mg/dL) | | | |
| Creatinine | 0.3 ± 0.07 | 0.43 ± 0.23 | 0.21 ± 0.08 |
| (mg/dL) | | | |
| Globulin (g/dL) | 2.65 ± 0.24 | 2.67 ± 0.79 | 2.8 ± 0.29 |
| Glucose | 103.0 ± 14.17 | 96.03 ± 17.72 | 93.50 ± 6.25^{‡} |
| (mg/dL) | | | |
| HDL-C (mg/dL) | 122.5 ± 13.18 | 237.03 ± 182.45* | 189.75 ± 20.19 |
| Potassium | 4.72 ± 0.2 | 5.47 ± 0.62† | 5.18 ± 0.12 |
| (mmol/L) | | | |
| LDL-C (mg/dL) | 4.75 ± 3.3 | 30.58 ± 29.29 | 117.00 ± 15.35^{‡} |
| Sodium | 144.0 ± 1.83 | 161.23 ± 19.04^{†} | 147.75 ± 0.5 |
| (mmol/L) | | | |
| NA:K | 30.5 ± 1.91 | 29.48 ± 1.07^{†} | 22.87 ± 11.35 |
| Phosphorus | 10.53 ± 0.68 | 8.13 ± 1.84 | 8.3 ± 0.86 |
| (mg/dL) | | | |
| Total Bilirubin | 1.09 ± 0.54 | 0.51 ± 0.8 | 0.44 ± 0.35 |
| (mg/dL) | | | |
| Protein (g/dL) | 5.48 ± 0.33 | 5.06 ± 1.23 | 6.0 ± 0.14 |
| TAG (mg/dL) | 154.75 ± 36.79 | 95.82 ± 42.76 | 46.0 ± 8.91 |

All comparisons made between week 0 (baseline), 6 (midpoint), and 12 (final) blood chemistries unless otherwise noted.
*Significant increase in measure from other diet group.
^{†}Comparisons made between week 4 blood chemistries.
^{‡}Comparisons made between week 10 blood chemistries. Analyzed via mixed-effects analysis with Sidak's multiple comparisons test between lean and DIO groups each week.
All data represented as means ± standard deviation and significance set at α = 0.05.

As demonstrated by the data presented in Tables 2-3, ferret blood chemistries showed elevated fasting glucose (midpoint p=0.0008, final p=0.0132) and reduced high-density lipoprotein cholesterol (HDL-C, midpoint p=0.0211) in the diet-induced obese ferrets compared to lean controls (Hein, et al. (2012) Vet. Rec. 171(9):218; Ohwada & Katahira (1993) Jikken Dobutsu 42(2):135-42). Total cholesterol (midpoint p=0.0174) was also elevated in the DIO model compared to lean. Western diets resulting in obesity can increase risks of non-alcoholic fatty liver disease, renal steatosis, inflammation and oxidative stress. DIO ferrets displayed elevated ALT (final p=0.0304), globulin (midpoint p<0.0001), phosphorus (midpoint p=0.0158), and total protein levels (midpoint p<0.0001, Table 2), indicative of early stage liver and kidney disease (Huynh & Laloi (2013) Vet. Clin. Exot. Anim. 16:121-144).

Visceral adiposity, assessed by massing total adipose tissue in the abdominal compartment upon necropsy (Table 4), was increased in DIO ferrets compared to lean, concordant with the observed increased waist circumference (FIG. 1D). No other organ had significant increases in mass.

TABLE 4

| Target | Lean | Obese |
|---|---|---|
| Heart (g) | 5.66 ± 0.99 | 6.00 ± 0.94 |
| Liver (g) | 35.31 ± 2.52 | 38.23 ± 4.13 |
| Spleen (g) | 11.83 ± 2.32 | 13.17 ± 3.37 |
| Left Kidney (g) | 3.17 ± 0.63 | 4.03 ± 1.08 |
| Right Kidney (g) | 3.11 ± 0.63 | 3.83 ± 0.84 |
| Kidney Differences (g) | 0.23 ± 0.21 | 0.53 ± 0.58 |
| Abdominal Fat (g) | 48.60 ± 12.87 | 91.23 ± 13.71* |
| Plasma leptin (pg/mL) | 105.2 ± 11.69 | 119.7 ± 13.23* |

All values reported as means ± standard error. Comparisons made using unpaired t-test after assessing for normality using the Shapiro-Wilk test. Significance set at α = 0.05.

Higher leptin levels were also observed in plasma from DIO ferrets compared to that of lean ferrets. Finally, accurate blood pressure readings in the ferret rely on invasive intraarterial methods or specialized equipment and were not performed (Olin, et al. (1997) Am. J. Vet. Res. 58(10):1065-9; van Zeeland, et al. (2017) Vet. J. 228:53-62). Taken together, the co-occurrence of visceral adiposity, hyperglycemia, and reduced HDL-C compared to lean control ferrets indicated the development of MetS in the DIO ferret model.

### Example 3: Disease Severity in Obese Ferrets

Obese ferrets, independent of viral strain, suffered increased risk of being symptomatic upon infection (Table 5) .

TABLE 5

| | | Cumulative | Total | |
|---|---|---|---|---|
| | | At-Risk | Symptomatic | Absolute |
| Virus | Diet | Days | Days | Risk |
| A/Memphis/ | Obese | 114 | 73 | 0.64 |
| 257/2019 | (n = 10) | | | |
| (H3N2) | Lean | 100 | 21 | 0.21 |
| | (n = 9) | | | |
| A/California/ | Obese | 88 | 37 | 0.42 |
| 04/2009 | (n = 8) | | | |
| (H1N1) | Lean | 112 | 18 | 0.16 |
| | (n = 8) | | | |
| A/Hong | Obese | 122 | 22 | 0.18 |
| Kong/1073/ | (n = 13) | | | |
| 1999 | Lean | 146 | 15 | 0.10 |
| (H9N2) | (n = 13) | | | |
| Total | Obese | 324 | 132 | 0.41 |
| | (n = 31) | | | |
| | Lean | 358 | 54 | 0.15 |
| | (n = 30) | | | |

¹Relative risk of symptoms if in obese group.

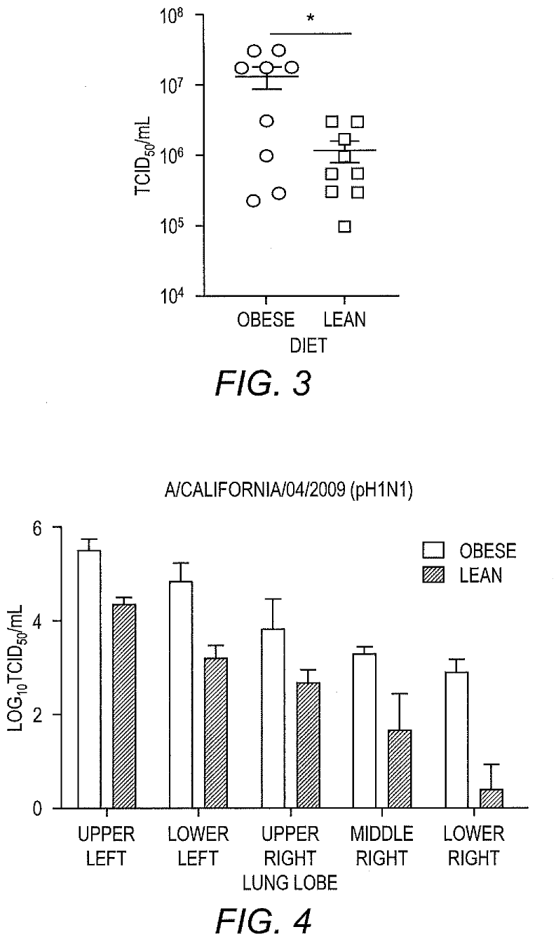

Compared to lean diet, an obese diet increases the odds of displaying clinical signs of influenza infection by 387% for all viruses (relative risk=2.70±0.87, p<0.0001), by 670% for H3N2 virus (relative risk=3.05±1.52, p<0.0001), by 379% for H1N1 virus (relative risk=2.62±1.65, p=0.0001), and by 192% for H9N2 virus (relative risk=1.76±1.48, p=0.0706). Over the course of infection, time- and diet-dependent increases in clinical score were observed for ferrets directly inoculated with H1N1 virus (days post-infection p<0.0001, diet p<0.0001, days post-infection×diet p<0.0001, FIGS. 2) and H3N2 virus (days post-infection p<0.0001, diet p<0.0001, days post-infection×diet p=0.0002, FIG. 2). In addition, increased viral titers were observed in the DIO ferrets early in infection (day 2) as compared to the lean ferrets (FIG. 3) and prolonged viral shedding was noted with symptomatic obese ferrets shedding 42% longer and asymptomatic obese ferrets shedding 104% longer than their lean counterparts. Moreover, there was an increase in viral spread to deep lung tissue in the DIO ferrets compared to those on the lean diet (FIG. 4). Given that the obese ferret model described herein mimics aspects of human obesity including increased influenza disease severity and prolonged viral shedding, this model is an excellent model system for studies of protection conferred by administered influenza vaccine.

What is claimed is:

1. A method for establishing an obese ferret model comprising feeding a ferret with a diet having at least 25% of carbohydrate content for a period of time to provide the obese ferret model.

2. The method of claim 1, wherein the obese ferret model exhibits at least one of visceral adiposity, hyperglycemia, or reduced high-density lipoprotein cholesterol compared to a control ferret.

3. The method of claim 1, wherein the obese ferret model does not develop insulinoma.

4. An obese ferret model established by the method of claim 1.

5. The obese ferret model of claim 4, wherein said obese ferret model exhibits at least one of visceral adiposity, hyperglycemia, or reduced high-density lipoprotein cholesterol levels compared to a control ferret.

6. The obese ferret model of claim 4, wherein said obese ferret model does not develop insulinoma.

7. A method for screening a substance for treating a respiratory infection comprising,
providing a test substance to an obese ferret model and a control ferret either before or after infecting the obese ferret model and a control ferret with an infectious agent that causes a respiratory infection; and
determining whether the test substance treats the respiratory infection.

8. The method of claim 7, wherein the obese ferret model exhibits at least one of visceral adiposity, hyperglycemia, or reduced high-density lipoprotein cholesterol levels compared to a control ferret.

9. The method of claim 7, wherein the infectious agent that causes a respiratory infection is a bacterium, fungus or virus.

10. The method of claim 9, wherein the virus is an influenza virus or coronavirus.
